(19) United States
(12) Patent Application Publication
(10) Pub. No.: US 2010/0305489 A1
(43) Pub. Date: Dec. 2, 2010

(54) CHITOSAN-BASED FIBER MATERIAL, ITS PREPARATION METHOD AND USE

(76) Inventors: Wanshun Liu, Shandong Province (CN); Baoqin Han, Shandong Province (CN); Gisheng Gu, Shanghai (CN)

Correspondence Address: JENKINS, WILSON, TAYLOR & HUNT, P. A., 3100 Tower Blvd., Suite 1200, DURHAM, NC, 27707, US

(21) Appl. No.: 12/599,769

(22) PCT Filed: Mar. 31, 2008
(86) PCT No.: PCT/CN2008/000636

§ 371 Date: Nov. 11, 2009

(30) Foreign Application Priority Data
May 13, 2007 (CN) 200710015743.6

(51) Int. Cl. A61F 13/00; C08B 37/08

(52) U.S. Cl. 602/43; 536/20

(57) ABSTRACT

The invention relates to a medical material, especially to a hemostatic and wound-healing water-soluble chitosan-based fiber material. The water-soluble chitosan-based fiber material comprises carboxymethyl chitosan fiber, hydroxypropyl chitosan fiber, hydroxyethyl chitosan fiber, carboxymethyl hydroxyethyl chitosan fiber or mixtures thereof.

### FIELD OF THE INVENTION

The invention relates to a medical material, more particularly to a hemostatic and wound-healing water-soluble chitosan-based fiber material, its preparation method and use.

### BACKGROUND OF THE INVENTION

Hemorrhage control, hemostasis and promotion of incision healing during and after clinical operation are necessary and important for shortening operation duration and reducing occurrence of complication after operation. Conventional hemostasis methods, such as ligation, suture and electrocoagulation, are commonly used for clinical is hemostasis and trauma hemostasis, but are not always effective for hemorrhage control, especially in the operation of parenchymal viscera containing many blood vessels. Therefore, many kinds of topical hemostatic materials have been put into clinical application according to particular clinical requirements, including glutin sponge, collagen sponge, fibrin glue and oxycellulose. Glutin sponge which has been applied in clinic for many years has a weaker hydrophilicity and a relatively poor hemostatic effect for massive hemorrhage. Collagen sponge which has a better hemostatic effect than glutin sponge is obtained from bovin achilles tendon and tendon, and therefore is exposed to a risk of spreading animal viral diseases. Fibrin glue has a wide application in the clinical operation hemostasis. Because fibrin glue components belong to blood products, however, fibrin glue is exposed to a risk of spreading antigenic diseases and viral diseases, and therefore is doubted for its application safety. The glutin sponge, collagen sponge and fibrin glue as mentioned above all belong to heterogeneous animal proteins, have certain immunogenicity and are exposed to a risk of spreading virus, while increasing chances of wound infection. Hemostatic materials made from oxycellulose have been applied to topical hemostasis in various clinical operations, such as neurosurgery, abdominal surgery, and thoracic surgery, with preference given to products sold by Johnson & Johnson, including Surgicel® absorbable hemostatic, Surgicel Nu-Knit® absorbable hemostatic, and Surgicel® Fibrllar absorbable hemostatic. However, the oxycellulose hemostatic material has a good hemostatic effect for slight blood exudation; in contrast, it exhibits slow hemostasis for serious blood exudation, and the effect is not quite ideal. Up to now a hemostatic material having a satisfactory hemostasis effect for serious blood exudation has not been developed. The hemostatic materials as mentioned above do not provide a promotive effect on incision healing or a satisfactory promotive effect on traumatic healing, while exerting a hemostatic effect.

Chitosan, a linear polysaccharide, is a positively charged, non-immunogenical and biodegradable product from chitin by removal of acetyl, which has many physiological activities, for example, promotion of wound healing, hemostasis and analgesic effect, antibacterial and bacteriostatic effect. The hemostatic effect of chitosan mainly results from the interaction between the charges of chitosan and erythrocyte and coagulation. However, the application of chitosan is greatly limited, especially in medical material, due to its dissolubility in water. Chitosan powder, chitosan membrane, chitosan sponge, and chitosan non-woven and woven fabrics obtained from chitosan degrade slowly in human body, and the complete degradation thereof takes over half a year. Many research shows that chitosan causes a systematic inflammatory reaction during degradation in the body. Therefore, hemostatic material and wound-healing material made from chitosan are only applicable to skin wound.

A carboxymethyl chitosan fiber and its preparation method and use are described in Chinese Patent No. ZL200410025721.4, the disclosure of which is incorporated herein by reference in its entirety. The polymeric monomer of the carboxymethyl chitosan fiber has a degree of substitution of carboxyl of 1 to 50%, imparting the fiber a high hygroscopicity while maintaining a fibrous structure and thus its dissolubility in water. However, the carboxymethyl chitosan fiber with a low degree of substitution takes a long time for degradation in body, and also would cause a systematic inflammatory reaction, and therefore is not applicable to body.

### DISCLOSURE OF THE INVENTION

The object of the invention is to provide a water-soluble chitosan-based fiber material and its preparation method and use to overcome the above drawbacks.

According to one aspect of the invention, there is provided a water-soluble chitosan-based fiber material, wherein the water-soluble chitosan-based fiber comprises carboxymethyl chitosan fiber, hydroxypropyl chitosan fiber, hydroxyethyl chitosan fiber, carboxymethyl hydroxyethyl chitosan fiber or mixtures thereof. The water-soluble chitosan-based fiber material according to the invention may be in the form of fiber, sliver, sponge, cotton wool, non-woven fabric, knitted fabric, braided fabric, woven fabric, etc.

According to another aspect of the invention, there is provided a method of preparing the water-soluble chitosan-based fiber material, comprising the following steps:

a water-insoluble chitosan fiber material is immersed in an aqueous solution of NaOH or KOH having a concentration of 20-50% by weight, preferably 30-45% by weight, a weight ratio of the water-insoluble chitosan fiber material to the aqueous solution being 1: (5-40), preferably 1: (10-25), and alkalized at a temperature ranging from −40° C. to room temperature for 2 to 72 hours, preferably 12 to 48 hours;

the alkalized water-insoluble chitosan-based fiber material is reacted with halogenated acetic acid, or halogenated ethanol, or propylene oxide, or halogenated propanol, or combination of halogenated acetic acid and halogenated ethanol in a reaction medium for a period of 5 to 36 hours, preferably 6 to 24 hours, to give a corresponding water-soluble chitosan fiber material;

wherein, the mole ratio of the water-insoluble chitosan-based fiber material to the alkaline to the halogenated acetic acid, or halogenated ethanol, or propylene oxide, or halogenated propanol, or combination of halogenated acetic acid and halogenated ethanol is 1: (10-40): (1.1-15), preferably 1: (20-40): (5-12).

According to another aspect of the invention, there is provided a method of preparing the water-soluble chitosan-based fiber material, comprising the following steps:

a water-soluble chitosan-based powder is dissolved in distilled water with stirring to form a viscous gel solution, and a minor amount of insoluble substance is removed by press filtering, giving a water-soluble chitosan-based spinning solution having a concentration of 2-10% by weight, preferably 5-8% by weight; the spinning solution is blown into a coagulation bath at a temperature of 30-45° C. via a spinning die, to form a water-soluble chitosan-based fiber; the fiber is drawn off by a capstan roll into a stretch bath, stretched by a draw roll and taken up, to produce a water-soluble chitosan-based fiber bundle; a dried water-soluble chitosan-based fiber is obtained by removal of organic solvent and drying; after finishing, cutting, packing and sterilizing, a sliver, sponge or cotton wool of the water-soluble chitosan-based fiber material is produced; or the water-soluble chitosan-based fiber material is further processed into the form of non-woven fabric, knitted fabric, braided fabric, woven fabric.

According to still another aspect, the invention further relates to the use of the water-soluble chitosan-based fiber material in producing drugs or medical devices for hemostasis, wound healing or prevention of tissue adhesion.

According to yet another aspect, the invention further relates to the use of drugs or medical devices produced from the water-soluble chitosan-based fiber material in hemostasis, promotion of wound healing and prevention of tissue adhesion before, during and after clinical operation.

The water-soluble chitosan-based fiber material according to the invention has all the effects of hemostasis, promotion of wound-healing and prevention of tissue adhesion; is capable of swelling gradually by absorbing water and dissolving gradually to form a gel solution in the physiological pH condition, being effective for topical sticking and exerting much better effects of hemostasis, wound-healing and prevention of tissue adhesion; and is free of immunogenicity in body, degrades in a short time, has a good biocompatibility, is safe in clinical application and suitable for hemostasis in body, wound healing and prevention of tissue adhesion. The water-soluble chitosan-based fiber material according to the invention in the form of sliver, sponge, cotton wool, non-woven fabric, knitted fabric, braided fabric, woven fabric has a certain mechanical strength, is convenient to handle in clinical operation, adheres strongly, and could exert its effects better and be more suitable for clinical application.

### PREFERRED EMBODIMENTS OF THE INVENTION

In the water-soluble chitosan-based fiber material according to the invention, the water-soluble chitosan-based fiber is selected from carboxymethyl chitosan fiber, hydroxypropyl chitosan fiber, hydroxyethyl chitosan fiber, carboxymethyl hydroxyethyl chitosan fiber or mixtures thereof.

More specifically, in the water-soluble chitosan-based fiber material according to the invention, the carboxymethyl chitosan may have the following structure:

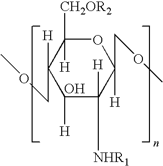

wherein, R₁ represents H, —COCH₃, —CH₂COONa or —CH₂COOK; R₂ represents H, —CH₂COONa or —CH₂COOK, and the degree of substitution of carboxymethyl is more than 60%. The carboxymethyl chitosan fiber is preferably sodium carboxymethyl chitosan fiber or potassium carboxymethyl chitosan fiber.

In the water-soluble chitosan-based fiber material according to the invention, the hydroxypropyl chitosan may have the following structure:

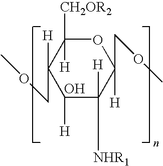

wherein, R₁ represents H, —COCH₃ or —CH₂CHOHCH₃; R₂ represents H or —CH₂CHOHCH₃, and the degree of substitution of hydroxypropyl is more than 60%.

In the water-soluble chitosan-based fiber material according to the invention, the hydroxyethyl chitosan may have the following structure:

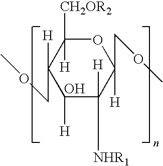

wherein, R₁ represents H, —COCH₃ or —CH₂CH₂OH; R₂ represents H or —CH₂CH₂OH, and the degree of substitution of hydroxyethyl is more than 60%.

In the water-soluble chitosan-based fiber material according to the invention, the carboxymethyl hydroxyethyl chitosan may have the following structure:

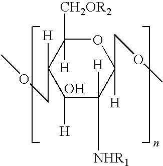

wherein, R₁ represents H, —COCH₃, —CH₂COONa, —CH₂COOK or —CH₂CH₂OH; R₂ represents H, —CH₂COONa, —CH₂COOK or —CH₂CH₂OH, and the total degree of substitution of carboxymethyl and to hydroxyethyl is more than 60%.

The water-soluble chitosan-based fiber material according to the invention may be prepared by a conventional method, starting from a water-soluble chitosan-based powder, such as water-soluble carboxymethyl chitosan, hydroxypropyl chitosan, hydroxyethyl chitosan, carboxymethyl hydroxyethyl chitosan or mixtures thereof. For example, the powder of water-soluble carboxymethyl chitosan, hydroxypropyl chitosan, hydroxyethyl chitosan, carboxymethyl hydroxyethyl chitosan or mixtures thereof is dissolved in distilled water with stirring at room temperature and left to stand, to form a viscous gel solution; the solution is press filtered in a filter by using a superposition of 200 mesh, 300 mesh, 400 mesh, 500 mesh silk to remove a minor amount of insoluble substances; the filtrate is degassed under vacuum to give a clear gel solution, that is water-soluble chitosan-based spinning solution. The spinning solution is blown into a coagulation bath via a spinning die, to form a water-soluble chitosan-based fiber. The fiber is drawn off by a capstan roll into a stretch bath, stretched by a draw roll and then taken up. The resulting water-soluble chitosan-based fiber bundle is subjected to centrifugal drying to remove organic solvents, and then dried at a temperature of 50-70° C., thereby obtaining a dried water-soluble chitosan-based fiber; after finishing, cutting, packing and sterilizing, a fiber sliver, sponge or cotton wool of the water-soluble chitosan-based fiber material is produced.

The water-soluble chitosan may be in a concentration of 2-10% by weight, preferably 5-8% by weight in the spinning solution. The coagulation bath and stretch bath may be one or two of ethanol and acetone, or an aqueous solution thereof in a concentration of more than 90% by weight. The temperature of the coagulation bath may be between 30° C. and 45° C. ⁶⁰Co sterilization or ethylene oxide sterilization is used for the sterilization.

The water-soluble chitosan-based fiber material obtained as above may be further processed into the form of non-woven fabric, knitted fabric, braided fabric, woven fabric by a method known to a person skilled in the art.

In the invention, non-woven fabric is a sheet, fiber web or wadding made from oriented or randomly oriented fiber bonded together by friction, cohesion or adhesion, or combination thereof. Woven fabric is produced by interlacing regularly two sets of yarns aligned respectively in the traverse and machine direction in a loom. Knitted fabric is produced by intertwining yarns or filaments in a series of loops pulled through each other by knitting needle. Braided fabric is produced by interweaving yarns or threads.

In another aspect, the water-soluble chitosan-based fiber material according to the invention may be prepared from a water-insoluble chitosan-based fiber by a method comprising the following steps: a water-insoluble chitosan-based fiber is immersed in an aqueous solution of NaOH or KOH having a concentration of 20-50% by weight in a weight ratio of 1:(5-40), and alkalized at a temperature ranging from −40° C. to room temperature for 2 to 72 hours; the alkalized water-insoluble chitosan-based fiber is transferred into a reaction medium and a reaction reagent, halogenated acetic acid, or halogenated ethanol, or propylene oxide, or halogenated propanol, or combination of halogenated acetic acid and halogenated ethanol, is added with agitating at room temperature, the mole ratio of the water-insoluble chitosan-based fiber material to the base to the halogenated acetic acid, or halogenated ethanol, or propylene oxide, or halogenated propanol, or combination of halogenated acetic acid and halogenated ethanol being 1: (10-40): (1.1-15); after reaction for a period of 5 to 36 hours, the resulting solid is washed by a washing solution until neutral, dehydrated and freed of excess liquids, and then is dried in a vacuum drier containing P₂O₅, subjected to evaporation to remove residual organic solvent; thereafter finishing, cutting, packing and sterilizing are carried out.

A water-insoluble chitosan fiber non-woven fabric, chitosan fiber, chitosan fiber woven fabric, chitosan fiber braided fabric can all be referred to as water-insoluble chitosan-based fiber. In the method according to the invention, the water-insoluble chitosan-based fiber may be one of chitosan fiber, chitosan fiber non-woven fabric, chitosan fiber knitted fabric, chitosan fiber braided fabric and chitosan fiber woven fabric.

The reaction medium may be one, two or three of methanol, ethanol, isopropanol, butanol, acetone, dimethylsulfoxide, tetrahydrofuran and dioxane, or an aqueous solution thereof.

The reaction reagent may be one of halogenated acetic acid, or one of halogenated ethanol, or propylene oxide, or one of halogenated propanol, or one of combination of halogenated acetic acid and halogenated ethanol. The halogenated acetic acid is chloracetic acid, bromacetic acid or iodoacetic acid. The halogenated ethanol is chlorethanol or bromethanol. The halogenated propanol is 1-chloro-2-propanol or 1-bromo-2-propanol. The combination of halogenated acetic acid and halogenated ethanol is chloracetic acid and chlorethanol, bromacetic acid and chlorethanol, chloracetic acid and bromethanol, or bromacetic acid and bromethanol.

The washing solution may be one, two or three of methanol, ethanol, isopropanol, butanol and acetone, or an aqueous solution thereof having a concentration of more than 75% by weight.

⁶⁰Co sterilization or ethylene oxide sterilization may be used for sterilizing.

The aqueous solution of the alkaline may have a concentration of 20-50% by weight, preferably 30-45% by weight. The weight ratio of the water-insoluble chitosan-based fiber to the aqueous solution may be 1: (5-40), preferably 1: (10-25). The alkalization may be performed at a temperature of −40° C. to room temperature for a period of 2-72 hours, preferably 12-48 hours. The mole ratio of the water-insoluble chitosan-based fiber, the alkaline and the reaction reagent is 1: (10-40): (1.1-15), preferably 1: (20-40): (5-12). The reaction may be performed for a period of 5-36 hours, preferably 6-14 hours, at room temperature.

In the method according to the invention, all the water-insoluble chitosan-based fiber, in the molecular structure of which the mole percentage of deacetylated glycosyl is between 0 and 100% of the total glycosyl, may achieve the same effects. Such fiber is

The degree of substitution of carboxymethyl for the water-soluble carboxymethyl chitosan fiber according to the invention depends on amount of the reaction reagent in mole, reaction time and temperature during the reaction, and the substitution of carboxymethyl mainly occurs on the position of R₂, while partly on the position of R₁. The reaction condition as recited above should be selected in such a way that a degree of substitution of carboxymethyl for the water-soluble carboxymethyl chitosan fiber more than 60% is achieved eventually.

The degree of substitution of hydroxypropyl for the water-soluble hydroxypropyl chitosan fiber according to the invention is depends on amount of the reaction reagent in mole, reaction time and temperature during the reaction, and the substitution of hydroxypropyl mainly occurs on the position of R₂, while partly on the position of R₁. The reaction condition as recited above should be selected in such a way that a degree of substitution of hydroxypropyl for the water-soluble hydroxypropyl chitosan fiber more than 60% is achieved eventually.

The degree of substitution of hydroxyethyl for the water-soluble hydroxyethyl chitosan fiber according to the invention depends on amount of the reaction reagent in mole, reaction time and temperature during the reaction, and the substitution of hydroxyethyl mainly occurs on the position of R₂, while partly on the position of R₁. The reaction condition as recited above should be selected in such a way that a degree of substitution of hydroxyethyl for the water-soluble hydroxyethyl chitosan fiber more than 60% is achieved eventually.

The degree of substitution of carboxymethyl and hydroxyethyl for the water-soluble carboxymethyl hydroxyethyl chitosan fiber according to the invention depends on amount of the reaction reagent in mole, reaction time and temperature during the reaction, and the substitution of carboxymethyl and hydroxyethyl mainly occurs on the position of R₂, while partly on the position of R₁. The reaction condition as recited above should be selected in such a way that a degree of substitution of carboxymethyl and hydroxyethyl for the water-soluble carboxymethyl hydroxyethyl chitosan fiber more than 60% is achieved eventually.

The water-soluble chitosan-based fiber material according to the invention feels soft, looks white, and has a good water solubility.

The following examples are given to explain the invention in more details. It is to be noted that numerous other modifications can be made by a person skilled in the art according to the invention described herein without departing from the scope of the invention. In the following examples, all temperature is in Celsius without calibration. And, unless otherwise indicated, all percentages of degree of substitution are based on mole, and the mole percentage is defined to be an average mole number of carboxymethyl or hydroxypropyl or hydroxyethyl or carboxymethyl and hydroxyethyl linked by each glycosyl. And other percentages are based on weight.

In the following examples, carboxymethyl chitosan sodium is produced by Boyite Biomaterial Company, Qingdao; and chitosan fiber, chitosan fiber non-woven fabric and chitosan fiber knitted fabric are commercially available from University of Donghua, Shanghai.

### Example 1

### Preparation of a Water-Soluble Carboxymethyl Chitosan Fiber Non-Woven Fabric

12 g of chitosan fiber non-woven fabric having an average weight of 20.5 mg/cm² is weighed into a 500 ml wide-necked flask, and 200 g of 45% aqueous KOH solution is added with stirring, so that chitosan fiber non-woven fabric is fully immersed into the alkaline solution. The flask with the content therein is placed at a temperature of −20° C. for 24 hours and the content is alkalized. The resulting alkalized chitosan fiber non-woven fabric is transferred into a 500 ml wide-necked flask, and 180 g of 55% aqueous acetone solution is added as a reaction medium, and 85 g of 50% monochloroacetic acid solution in ethanol is added as a reaction reagent with agitating. After reaction for 9 hours at room temperature, carboxymethyl chitosan potassium fiber non-woven fabric is obtained. The carboxymethyl chitosan potassium fiber non-woven fabric is washed sufficiently with 60% aqueous acetone solution till neutral, dehydrated with anhydrous acetone, then subjected to pressing or centrifugal drying to remove excess liquid, and dried in a vacuum drier containing P₂O₅. Then residual organic solvent is removed from the dried product by evaporation at a temperature of 50° C.; thereby obtaining a carboxymethyl chitosan fiber non-woven fabric having a degree of substitution of carboxymethyl of 92%. Thereafter finishing, cutting, packing and ⁶⁰Co sterilization are carried out, to give a water-soluble chitosan-based fiber material.

### Example 2

### Preparation of a Water-Soluble Carboxymethyl Chitosan Fiber

5 g of chitosan fiber having a length of 10 cm is weighed into a 500 ml wide-necked flask, and 160 g of 30% aqueous NaOH solution is added with stirring, so that chitosan fiber is fully immersed into the alkaline solution. The flask with the content therein is placed at room temperature for 48 hours and the content is alkalized. The resulting alkalized chitosan fiber is transferred into a 500 ml wide-necked flask, and 160 g of methanol is added as a reaction medium, and 46 g of 50% monochloroacetic acid solution in methanol is added as a reaction reagent with agitating. After reaction for 8 hours at room temperature, carboxymethyl chitosan sodium fiber is obtained. The carboxymethyl chitosan sodium fiber is washed sufficiently with methanol till neutral, then subjected to pressing or centrifugal drying to remove excess liquid, and dried in a vacuum drier containing P₂O₅. Then residual organic solvent is removed from the dried product by evaporation at a temperature of 50° C.; thereby obtaining a carboxymethyl chitosan fiber having a degree of substitution of carboxymethyl of 96%. Thereafter finishing, cutting, packing and propylene oxide sterilization are carried out, to give a water-soluble chitosan-based fiber material.

The water-soluble chitosan-based fiber material prepared according to the invention may be in the form of sliver, sponge or cotton wool of carboxymethyl chitosan fiber.

### Example 3

### Preparation of a Water-Soluble Carboxymethyl Chitosan Fiber Knitted Fabric

10 g of chitosan fiber knitted fabric having an average weight of 15.6 mg/cm² is weighed into a 500 ml wide-necked flask, and 200 g of 40% aqueous NaOH solution is added with stirring, so that chitosan fiber knitted fabric is fully immersed into the alkaline solution. The flask with the content therein is placed at a temperature of 0-4° C. for 48 hours and the content is alkalized. The resulting alkalized chitosan fiber knitted fabric is transferred into a 500 ml wide-necked flask, and 135 g of 90% aqueous ethanol solution and 50 g of dimethyl sulfoxide are added as reaction medium, and 70 g of 50% monochloroacetic acid solution in ethanol is added as a reaction reagent with agitating. After reaction for 10 hours at room temperature, carboxymethyl chitosan sodium fiber knitted fabric is obtained. The carboxymethyl chitosan sodium fiber knitted fabric is washed sufficiently with 75% aqueous ethanol solution till neutral, dehydrated with anhydrous ethanol, then subjected to pressing or centrifugal drying to remove excess liquid, and dried in a vacuum drier containing P₂O₅. Then residual organic solvent is removed from the dried product by evaporation at a temperature of 70° C.; thereby obtaining a carboxymethyl chitosan fiber knitted fabric having a degree of substitution of carboxymethyl of 90%. Thereafter finishing, cutting, packing and propylene oxide sterilization are carried out, to give a water-soluble chitosan-based fiber material.

The reaction reagent used in Examples 1 to 3 may be one of halogenated acetic acid, namely chloracetic acid, bromacetic acid and idoacetic acid. All of them may achieve the same effects.

### Example 4

### Preparation of a Water-Soluble Hydroxypropyl Chitosan Fiber Non-Woven Fabric

10 g of chitosan fiber non-woven fabric having an average weight of 20.5 mg/cm² is weighed into a 500 ml wide-necked flask, and 200 g of 30% aqueous NaOH solution is added with stirring, so that chitosan fiber non-woven fabric is fully immersed into the alkaline solution. The flask with the content therein is placed at a temperature of −20° C. for 60 hours and the content is alkalized. The resulting alkalized chitosan fiber non-woven fabric is transferred into a 1000 ml stainless steel reaction bottle with a sealing cap, and 150 g of isopropanol, 25 g of dimethyl sulfoxide and 25 g of dioxane are added as reaction medium, and 45 g of propylene oxide is added as a reaction reagent with agitating. The reaction bottle is sealed with the cap. After reaction for 24 hours with agitating at room temperature, hydroxypropyl chitosan fiber non-woven fabric is obtained. The hydroxypropyl chitosan fiber non-woven fabric is washed sufficiently with 80% aqueous acetone solution till neutral, dehydrated with anhydrous acetone, then subjected to pressing or centrifugal drying to remove excess liquid, and dried in a vacuum drier containing P₂O₅. Then residual organic solvent is removed from the dried product by evaporation at a temperature of 50° C.; thereby obtaining a hydroxypropyl chitosan fiber non-woven fabric having a degree of substitution of hydroxypropyl of 86%. Thereafter finishing, cutting, packing and ⁶⁰Co sterilization are carried out, to give a water-soluble chitosan-based fiber material.

In the example, the reaction agent may be one of propylene oxide and halogenated propanol, namely propylene oxide, 1-chloro-2-propanol and 1-bromo-2-propanol. All of them may achieve the same effects.

### Example 5

### Preparation of a Water-Soluble Hydroxyethyl Chitosan Fiber Non-Woven Fabric

10 g of chitosan fiber non-woven fabric having an average weight of 20.5 mg/cm² is weighed into a 500 ml wide-necked flask, and 200 g of 45% aqueous KOH solution is added with stirring, so that chitosan fiber non-woven fabric is fully immersed into the alkaline solution. The flask with the content therein is placed at a temperature of −20° C. for 48 hours and the content is alkalized. The resulting alkalized chitosan fiber non-woven fabric is transferred into a 1000 ml stainless steel reaction bottle with a sealing cap, and 160 g of acetone, 20 g of dimethyl sulfoxide and 20 g of tetrahydrofuran are added as reaction medium, and 55 g of monochloroethanol is added as a reaction reagent with agitating. The reaction bottle is sealed with the cap. After reaction for 36 hours with agitating at room temperature, hydroxyethyl chitosan fiber non-woven fabric is obtained. The hydroxyethyl chitosan fiber non-woven fabric is washed sufficiently with 85% aqueous acetone solution till neutral, dehydrated with acetone, then subjected to pressing or centrifugal drying to remove excess liquid, and dried in a vacuum drier containing P₂O₅. Then residual organic solvent is removed from the dried product by evaporation at a temperature of 50° C.; thereby obtaining a hydroxyethyl chitosan fiber non-woven fabric having a degree of substitution of hydroxyethyl of 91%. Thereafter finishing, cutting, packing and ethylene oxide sterilization are carried out, to give a water-soluble chitosan-based fiber material.

In the example, the reaction agent may be one of halogenated ethanol, namely chloroethanol and bromoethanol. Both of them may achieve the same effects.

### Example 6

### Preparation of a Water-Soluble Carboxymethyl Hydroxyethyl Chitosan Fiber Non-Woven Fabric

10 g of chitosan fiber non-woven fabric having an average weight of 20.5 mg/cm² is weighed into a 500 ml wide-necked flask, and 200 g of 45% aqueous KOH solution is added with stirring, so that chitosan fiber non-woven fabric is fully immersed into the alkaline solution. The flask with the content therein is placed at a temperature of −20° C. for 48 hours and the content is alkalized. The resulting alkalized chitosan fiber non-woven fabric is transferred into a 1000 ml stainless steel reaction bottle with a sealing cap, and 160 g of acetone, 20 g of dimethyl sulfoxide and 20 g of tetrahydrofuran are added as reaction medium, and 46 g of monochloroacetic acid solution in ethanol and 30 g of monochloroethanol are added as reaction reagents with agitating. The reaction bottle is sealed with the cap. After reaction for 48 hours with agitating at room temperature, carboxymethyl hydroxyethyl chitosan fiber non-woven fabric is obtained. The carboxymethyl hydroxyethyl chitosan fiber non-woven fabric is washed sufficiently with 85% aqueous acetone solution till neutral, dehydrated with acetone, then subjected to pressing or centrifugal drying to remove excess liquid, and dried in a vacuum drier containing P₂O₅. Then residual organic solvent is removed from the dried product by evaporation at a temperature of 50° C.; thereby obtaining a carboxymethyl hydroxyethyl chitosan fiber non-woven fabric having a degree of substitution of carboxymethyl and hydroxyethyl of 94%. Thereafter finishing, cutting, packing and ethylene oxide sterilization are carried out, to give a water-soluble chitosan-based fiber material.

### Example 7

120 g of water-soluble carboxymethyl chitosan sodium powder having a degree of substitution of carboxymethyl more than 60% is weighed into a 3000 ml clean stainless steel container, and 2000 ml deionized water is added. The mixture is stirred at room temperature and left to stand, allowing a complete dissolution of the carboxymethyl chitosan sodium powder to form a viscous gel solution. The solution is press filtered in a filter by using a superposition of 200 mesh, 300 mesh, 400 mesh, 500 mesh silk to remove a minor amount of insoluble substance; the filtrate is degassed under vacuum to give a clear gel solution of carboxymethyl chitosan, that is water-soluble chitosan spinning solution. The spinning solution is blown into a coagulation bath of 95% aqueous ethanol solution at a temperature of 40-45° C. via a spinning die, to form carboxymethyl chitosan fiber. The fiber is drawn off by a capstan roll into a stretch bath of 95% aqueous ethanol solution, stretched by a draw roll and then taken up. The resulting carboxymethyl chitosan fiber bundle is subjected to centrifugal drying to remove organic solvents, and then dried at a temperature of 50-70° C., thereby obtaining carboxymethyl chitosan fiber. After finishing, cutting, packing and ethylene oxide sterilization, a sliver, sponge or cotton wool of the carboxymethyl chitosan fiber is produced.

In Example 7, the carboxymethyl chitosan sodium may be replaced with carboxymethyl chitosan potassium, hydroxypropyl chitosan or hydroxyethyl chitosan. All of them are referred to as water-soluble chitosan-based material, which has a degree of substitution of carboxymethyl, hydroxypropyl or hydroxyethyl more than 60%, and may achieve the same effects. The concentration of the water-soluble chitosan-based fiber material in the spinning solution may be 2-10% by weight, preferably 5-8%. The coagulation bath and stretch bath may be one or both of ethanol and acetone, or an aqueous solution thereof in a concentration more than 90% by weight. The temperature of the coagulation bath may be 30-45° C. Either ⁶Co sterilization or ethylene oxide sterilization may be utilized.

### Example 8

The carboxymethyl chitosan fiber obtained in Example 7 is subjected to opening, carding, cutting off, lapping, backing, fleece forming, cutting and drying. The resulting carboxymethyl chitosan fiber non-woven fabric is subjected to finishing, cutting, packing and ethylene oxide sterilization, to produce a non-woven fabric of water-soluble chitosan-based fiber material.

In Example 8, the carboxymethyl chitosan fiber may be replaced with hydroxypropyl chitosan fiber or hydroxyethyl chitosan fiber, and all of them may achieve the same effects. The non-woven fabric of water-soluble chitosan-based fiber material refers to carboxymethyl chitosan fiber non-woven fabric, hydroxypropyl chitosan fiber non-woven fabric or hydroxyethyl chitosan fiber non-woven fabric.

### Example 9

The performances of the water-soluble chitosan-based fiber material with regard to hemostasis, wound healing and prevention of tissue adhesion after operation are tested and evaluated as follows:

A liver tissue defect model is produced by using a rat as a test animal, and the respective effect of an oxidized regenerated cellulose gauze (a product by Johnson & Johnson), a water-soluble carboxymethyl chitosan fiber non-woven fabric obtained in example 1, a water-soluble hydroxypropyl chitosan fiber non-woven fabric obtained in example 4 and a water-soluble hydroxyethyl chitosan fiber non-woven fabric obtained in example 5 with regard to hemostasis, wound-healing and prevention of tissue adhesion after operation for the liver tissue defect is evaluated and compared. Before the test, all of the materials are cut into pieces of 1 cm*1 cm in a sterile environment. 60 SD male rats weighed 250 g±20 g are chosen and divided randomly into negative control group (no material), oxidized regenerated cellulose gauze control group (shortened as oxidized cellulose gauze group, in which oxidized regenerated cellulose gauze is used as the material), water-soluble chitosan-based material test group No. 1 (shortened as carboxymethyl chitosan fiber non-woven fabric group, in which carboxymethyl chitosan fiber non-woven fabric is used as the material), water-soluble chitosan-based material test group No. 2 (shortened as hydroxypropyl chitosan fiber non-woven fabric group, in which hydroxypropyl chitosan fiber non-woven fabric is used as the material) and water-soluble chitosan-based material test group No. 3 (shortened as hydroxyethyl chitosan fiber non-woven fabric group, in which hydroxyethyl chitosan fiber non-woven fabric is used as the material), with 12 rats in each group. All the rats are given an anaesthesia with pentobarbital sodium intraperitoneal injection in a dosage of 30 mg/kg. And the anaesthetized rats are laparotomized by making an incision with a length of about 3 cm on median abdomen, through which left lobe of liver is externalized under sterile condition, and a liver tissue of 5 mm in length*5 mm in width*2 mm in thickness is removed downside from median liver by using a operating forceps, thereby causing a liver tissue defect. After 5 seconds of haemorrhage from the removal of the liver to tissue, a medical gauze is used to absorb blood, and the corresponding material of 1 cm*1 cm is applied immediately. Three layers of the oxidized cellulose gauze (weight 61-63 g), two layers of carboxymethyl chitosan fiber non-woven fabric (weight 59-61 g), two layers of hydroxypropyl chitosan fiber non-woven fabric (weight 61-63 g), two layers of hydroxyethyl chitosan fiber non-woven fabric (weight 60-62 g) are applied respectively. Thereafter, 10 g poise is placed on the materials and remains for 20 seconds, then traumatic bleeding is observed for 40 seconds. The above procedure is repeated and bleeding is recorded. After 3 minutes, the incision is sewed and disinfected by iodophor. The rats are raised in groups and the effects on traumatic healing for animal liver tissue defect and tissue adhesion are observed respectively on days 7 and 14.

The results are summarized in tables 1 and 2. It is shown that the three types of water-soluble chitosan-based fiber materials and the oxidized regenerated cellulose gauze, compared to the negative control group, have notable effects of hemostasis, promotion of traumatic healing and prevention of tissue adhesion; furthermore, the three types of water-soluble chitosan-based fiber materials exert better effects by reducing significantly hemostasis time and traumatic healing time, and preventing tissue adhesion after operation.

TABLE 1

| | test animal | Hemostasis time |
|---|---|---|
| group | number | (seconds) |
| negative comparison | 12 | >180 |
| oxidized cellulose gauze | 12 | 80-120 |
| Carboxymethyl chitosan fiber | 12 | ≦20 |
| non-woven fabric | | |
| hydroxypropyl chitosan fiber | 12 | ≦30 |
| non-woven fabric | | |
| hydroxyethyl chitosan fiber | 12 | ≦30 |
| non-woven fabric | | |

TABLE 2
evaluation on the performances of the water-soluble
chitosan-based fiber material with regard to promotion of traumatic
healing and prevention of tissue adhesion after operation

| | test results after 7 days | | test results after 14 days | |
|---|---|---|---|---|
| | animal | traumatic healing | animal | traumatic healing |
| group | number | after 7 days | number | after 14 days |
| negative | 3 | around 20% | 4 | around 20% |
| control* | | traumatic healing and | | traumatic healing |
| | | serious tissue | | and serious tissue adhesion |
| | | adhesion | | |
| oxidized | 6 | the material | 6 | the material |
| cellulose gauze | | substantially | | substantially |
| | | degraded, forming | | degraded, forming |
| | | purple black gel, | | purple black gel on |
| | | around 40%-50% | | the wound, around |
| | | traumatic healing, | | 80%-90% traumatic |
| | | and presence of tissue | | healing, and |
| | | adhesion | | presence of tissue |
| | | | | adhesion |
| Carboxymethyl | 6 | the material | 6 | the material |
| chitosan fiber | | degraded, around | | degraded, complete |
| non-woven fabric | | 70%-80% traumatic | | traumatic healing, |
| | | healing, and no tissue | | and no tissue |
| | | adhesion | | adhesion |
| hydroxypropyl | 6 | the material | 6 | the material |
| chitosan fiber | | degraded, around | | degraded, complete |
| non-woven fabric | | 70%-80% traumatic | | traumatic healing, |
| | | healing, and no tissue | | and no tissue |
| | | adhesion | | adhesion |
| hydroxyethyl | 6 | the material | 6 | the material |
| chitosan fiber | | degraded, around | | degraded, complete |
| non-woven fabric | | 70%-80% traumatic | | traumatic healing, |
| | | healing, and no tissue | | and no tissue |
| | | adhesion | | adhesion |

*5 rats in the negative control group died within 24 hours of operation

A drug or medical device can be made from the water-soluble chitosan-based fiber material according to the invention and applied to clinical abdomen operation, thorax operation, bone operation, gynecological operation, microsurgery operation, burn surgery, plastic operation, etc. The water-soluble chitosan-based fiber material may exert effectively its effects of hemostasis, prevention of traumatic healing and prevention of tissue adhesion after operation, when being applied on bleeding location with gentle pressure. Especially, the water-soluble chitosan-based fiber material according to the invention has a good biocompatibility, won't cause a systematic inflammatory reaction, has a short degradation in body, has good effects of hemostasis, wound healing and prevention of tissue adhesion after operation, and therefore is applicable to operation trauma.

The examples as described above show that the water-soluble chitosan-based fiber material according to the invention is useful in producing drug or medical device which is applicable for hemostasis, promotion of wound healing or prevention of tissue adhesion after operation.

1. A water-soluble chitosan-based fiber material, wherein the water-soluble chitosan-based fiber comprises carboxymethyl chitosan fiber, hydroxypropyl chitosan fiber, hydroxyethyl chitosan fiber, carboxymethyl hydroxyethyl chitosan fiber or a mixture thereof.

2. The water-soluble chitosan-based fiber material according to claim 1, wherein the carboxymethyl chitosan has the following structure: 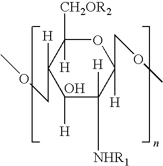
wherein, R₁ represents H, —COCH₃, —CH₂COONa or —CH₂COOK; R₂ represents H, —CH₂COONa or —CH₂COOK, and the degree of substitution of carboxymethyl is more than 60%.

3. The water-soluble chitosan-based fiber material according to claim 1, wherein the hydroxypropyl chitosan has the following structure: 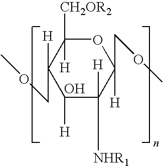
wherein, R₁ represents H, —COCH₃ or —CH₂CHOHCH₃; R₂ represents H or —CH₂CHOHCH₃, and the degree of substitution of hydroxypropyl is more than 60%.

4. The water-soluble chitosan-based fiber material according to claim 1, wherein the hydroxyethyl chitosan has the following structure: 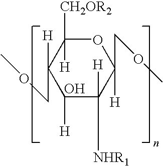
Wherein, R₁ represents H, —COCH₃ or —CH₂CH₂OH; R₂ represents H or —CH₂CH₂OH, and the degree of substitution of hydroxyethyl is more than 60%.

5. The water-soluble chitosan-based fiber material according to claim 1, wherein the carboxymethyl hydroxyethyl chitosan has the following structure: 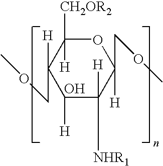
Wherein, R₁ represents H, —COCH₃, —CH₂COONa, —CH₂COOK or —CH₂CH₂OH; R₂ represents H, —CH₂COONa, —CH₂COOK or —CH₂CH₂OH, and the total degree of substitution of carboxymethyl and hydroxyethyl is more than 60%.

6. The water-soluble chitosan-based fiber material according to any one of claims 1-5 in the form of fiber, sliver, sponge, cotton wool, non-woven fabric, knitted fabric, braided fabric, woven fabric.

7. A method for preparing the water-soluble chitosan-based fiber material according to claim 1, comprising the following steps:
a water-insoluble chitosan fiber material is immersed and alkalized in an aqueous solution of NaOH or KOH having a concentration of 20-50% by weight, preferably 30-45% by weight, with the weight ratio of the water-insoluble chitosan fiber material to the aqueous solution being 1: (5-40), preferably 1: (10-25), at a temperature of from −40° C. to room temperature for 2 to 72 hours, preferably 12 to 48 hours;
the alkalized water-insoluble chitosan-based fiber material is reacted with halogenated acetic acid, or halogenated ethanol, or propylene oxide, or halogenated propanol, or combination of halogenated acetic acid and halogenated ethanol in a reaction medium for a period of 5 to 36 hours, preferably 6 to 24 hours, to give the corresponding water-soluble chitosan fiber material,
wherein the mole ratio of the water-insoluble chitosan-based fiber material to the alkaline to the halogenated acetic acid, or halogenated ethanol, or propylene oxide, or halogenated propanol, or combination of halogenated acetic acid and halogenated ethanol is 1: (10-40): (1.1-15), preferably 1: (20-40): (5-12).

8. The method according to claim 7, wherein the halogenated acetic acid is chloracetic acid, bromacetic acid or iodoacetic acid, the halogenated ethanol is chlorethanol or bromethanol, the halogenated propanol is 1-chloro-2-propanol or 1-bromo-2-propanol, and the combination of halogenated acetic acid and halogenated ethanol is chloracetic acid and chlorethanol, bromacetic acid and chlorethanol, chloracetic acid and bromethanol, or bromacetic acid and bromethanol.

9. The method according to claim 7, wherein the reaction medium is one or more of methanol, ethanol, isopropanol, butanol, acetone, dimethyl sulfoxide, tetrahydrofuran and dioxane, or an aqueous solution thereof.

10. The method according to claim 7, further comprising a step of washing the product with a washing solution till neutral.

11. The method according to claim 10, wherein the washing solution may be one, two or three of methanol, ethanol, isopropanol, butanol and acetone, or an aqueous solution thereof having a concentration of more than 75% by weight.

12. The method according to claim 7, further comprising a step of sterilization.

13. The method according to claim 12, wherein the sterilization is ⁶⁰Co sterilization or ethylene oxide sterilization.

14. The method according to any one of claims 7-13, wherein the water-insoluble chitosan-based fiber is in the form of chitosan fiber, chitosan fiber non-woven fabric, chitosan fiber knitted fabric, chitosan fiber braided fabric or chitosan fiber woven fabric, in the molecular structure of which the mole percentage of deacetylated glycosyl may be between 0 and 100% of the total glycosyl.

15. A method for preparing the water-soluble chitosan-based fiber material according to any one of claims 1-5, comprising the following steps:
a water-soluble chitosan-based powder is dissolved in distilled water to give a water-soluble chitosan-based spinning solution having a concentration of 2-10% by weight, preferably 5-8% by weight;
the spinning solution is blown into a coagulation bath at a temperature of 30-45° C. via a spinning die, to form a water-soluble chitosan-based fiber;
the fiber is drawn off by a capstan roll into a stretch bath, stretched by a draw roll and taken up, to produce a water-soluble chitosan-based fiber bundle;
a dried water-soluble chitosan-based fiber is obtained by removal of organic solvent and drying.

16. The method according to claim 15, further comprising steps of finishing, cutting, packing and sterilizing.

17. The method according to claim 15, furthering comprising a step of further processing the dried water-soluble chitosan-based fiber into a material in the form of fiber sliver, sponge, cotton wool, non-woven fabric, knitted fabric, braided fabric or woven fabric.

18. The method according to claim 15, wherein the water-soluble chitosan-based powder may be powder of carboxymethyl chitosan, hydroxypropyl chitosan, hydroxyethyl chitosan, carboxymethyl hydroxyethyl chitosan or a mixture thereof, and the degree of substitution of carboxymethyl, hydroxypropyl or hydroxyethyl is more than 60%.

19. The method according to claim 15, wherein the coagulation bath and the stretch bath may be one or both of ethanol and acetone, or an aqueous solution thereof having a concentration more than 90% by weight.

20. The method according to claim 16, wherein the sterilizing is ⁶⁰Co sterilization or ethylene oxide sterilization.

21. Use of the water-soluble chitosan-based fiber material according to any one of claims 1-5 in producing a drug or medical device for hemostasis, wound healing or prevention of tissue adhesion.

22. The use according to claim 21, wherein the drug or medical device is used for hemostasis, promotion of wound healing and prevention of tissue adhesion before, during and after clinical operation.
